(19) United States
(12) Patent Application Publication
(10) Pub. No.: US 2024/0050946 A1
(43) Pub. Date: Feb. 15, 2024

(54) Biosensor Chip

(71) Applicant: National Chung Cheng University, Chia-Yi (TW)

(72) Inventors: Lai-Kwan Chau, Chiayi City (TW); Chih-Hsien Wang, Tainan City (TW); Yue-Jin Cai, Chiayi City (TW)

(21) Appl. No.: 18/325,042

(22) Filed: May 29, 2023

(30) Foreign Application Priority Data
Aug. 10, 2022 (TW) 111130086

(51) Int. Cl. B01L 3/00; B82Y 15/00

(52) U.S. Cl. CPC B01L 3/502753; B01L 3/502707; B82Y 15/00

(57) ABSTRACT

The present disclosure provides a biosensor chip including: a top cover including a sample loading slot for injecting blood sample, a glue injection hole, and a vent hole; a plasma separation membrane overlapping the sample slot; a conjugate pad overlapping the plasma separation membrane; an optical fiber; and a bottom cover including a conjugate pad groove for setting the conjugate pad, an optical fiber channel for setting the optical fiber, a glue injection groove corresponding to the position of the glue injection hole in the top cover, and a waste liquid tank, wherein the conjugate pad groove and the optical fiber channel are connected, the glue injection groove overlaps with the optical fiber channel, the vent hole is located on the waste liquid tank, and the upper cover further includes a separation membrane groove where the plasma separation membrane is set, and a flow channel connecting the optical fiber channel and the waste liquid tank.

### BACKGROUND OF THE INVENTION

### 1. Field of the Invention

The present disclosure generally relates to biosensor chips, in particular to a biosensor chip combined with a plasma separation membrane and a conjugate pad, which is capable of separating plasma from blood sample and detecting the analyte in plasma by simply injecting a diluted blood sample into the chip.

### 2. Description of the Related Art

Recently, in order to easily and quickly detect and analyze specific components in biofluids, the use of biosensor chips targeting body fluid samples such as blood, urine, or saliva has gradually increased. Wherein, blood plays an important role in the detection of many diseases, so that blood becomes a commonly used sample for biosensor chips.

A fiber optic particle plasmon resonance (FOPPR) biosensor has been developed in recent years. The biosensor uses a fiber optic evanescent wave absorption configuration based on particle plasmon resonance (PPR) to absorb light waves to detect various biomarkers. For example, the FOPPR biosensor has excellent accuracy in the analysis of biomarkers such as cardiac troponin I (cTnI) in real serum sample, but in the analysis of blood sample, there are difficulties in quantitative analysis due to the influence of blood cells (red blood cells, white blood cells and platelets), etc.

Conventionally, when the FOPPR sensor chips were used to detect whole blood sample, plasma or serum must be separated from the whole blood sample before subsequent analysis. In the laboratory, a common method for pretreatment of a whole blood sample is to use a centrifuge to centrifuge the blood, extract the plasma for dilution, then mix the diluted plasma with the nanoparticles modified by a detection probe, and inject it into the FOPPR sensor chip for quantification analysis. However, the need of centrifugation steps and mixing steps results in inconvenience, which makes it difficult to realize the convenience of Lab-on-a-chip.

### SUMMARY OF THE INVENTION

In view of the above problems, the objective of the present disclosure is to provide a biosensor chip capable of analyzing blood sample directly without centrifugation steps, so as to omit the cumbersome centrifugation steps and mixing steps.

To achieve the foregoing objective, the present disclosure provides a biosensor chip, comprising: a top cover provided with a sample loading slot for injecting a blood sample, a glue injection hole, and a vent hole; a plasma separation membrane overlapping the sample loading slot of the top cover; a conjugate pad overlapping the plasma separation membrane; an optical fiber; and a bottom cover provided with a conjugate pad groove for disposing the conjugate pad, an optical fiber channel for disposing the optical fiber, a glue injection groove corresponding to a position of the glue injection hole in the top cover for disposing a glue, and a waste liquid tank, wherein, the conjugate pad groove is connected to the optical fiber channel, the glue injection groove is partially overlapped with the optical fiber channel, and the vent hole in the top cover is located above the waste liquid tank in the bottom cover, the top cover further includes a separation membrane groove for disposing the plasma separation membrane, and a flow channel for connecting the optical fiber channel and the waste liquid tank.

In a preferred embodiment of the present disclosure, the conjugate pad includes nanoparticles modified by a detection probe, which is capable of binding with an analyte molecule in the blood sample.

In a preferred embodiment of the present disclosure, the detection probe includes one selected from a group consisting of antibodies, nucleic acid aptamers, peptides, hormone receptors, lectins, carbohydrates, chemical recognition molecules, deoxyribonucleic acid, ribonucleic acid, and nucleic acid aptamers.

In a preferred embodiment of the present disclosure, the nanoparticles are gold nanoparticles. However, the material of the nanoparticles is not limited to gold, and may also include other colored nanoparticles.

In a preferred embodiment of the present disclosure, the analytes include DNA, RNA, protein, small molecule, or antibody.

In a preferred embodiment of the present disclosure, the optical fiber is partially unclad to expose a segment of optical fiber core, where a surface of the optical fiber core is modified by a capture probe which is capable to capture the analyte molecule bound to the detection probe which is conjugated to the nanoparticle, and the analyte molecule bound to the detection probe which is conjugated to the nanoparticle forms a sandwich-like nanocomplex with the capture probe.

In a preferred embodiment of the present disclosure, the capture probe includes one selected from a group consisting of antibodies, nucleic acid aptamers, peptides, hormone receptors, lectins, carbohydrates, chemical recognition molecules, deoxyribonucleic acid, ribonucleic acid, and nucleic acid aptamers.

In a preferred embodiment of the present disclosure, the optical fiber core is disposed in the optical fiber corresponding to the position where the conjugate pad groove and the optical fiber channel are connected.

In a preferred embodiment of the present disclosure, the plasma separation membrane includes polysulfone, polyethersulfone, or glass fiber.

In a preferred embodiment of the present disclosure, the conjugate pad is made of nonabsorbable glass fiber.

In summary, the biosensor chip utilizes a plasma separation membrane to separate plasma from whole blood and utilizes a conjugate pad to stably preserve properties of the nanoparticles modified by the detection probe. Therefore, it is not necessary to centrifuge the blood sample and mix the diluted plasma with the nanoparticles modified by the detection probe before the detection, but simply inject the diluted whole blood sample directly into the sensor chip to achieve the effect of separating the plasma and mixing the nanoparticles modified by the detection probe in one step, so as to analyze the blood sample in a simpler and faster way.

### BRIEF DESCRIPTION OF THE DRAWINGS

The features and technical contents of the present disclosure will be further appreciated and understood with reference to the detailed description of preferred embodiments and accompanying drawings.

FIG. 1 is a stereoscopic diagram of a top cover and a plasma separation membrane of the biosensor chip in accordance with an embodiment of the present disclosure.

FIG. 2 is a stereoscopic diagram of a plasma separation membrane, an optical fiber, and a bottom cover of the biosensor chip in accordance with an embodiment of the present disclosure.

FIG. 3 is an enlarged view of the optical fiber in FIG. 2.

FIG. 4 is an assembly diagram of the biosensor chip in accordance with an embodiment of the present disclosure.

FIG. 5 is a decomposition diagram of the biosensor chip in accordance with an embodiment of the present disclosure.

FIG. 6 is a stereoscopic diagram of the biosensor chip in accordance with an embodiment of the present disclosure.

FIG. 7 is a schematic cross-sectional view taken along the V-V line in FIG. 6.

FIG. 8 is an operation diagram of the biosensor chip in accordance with an embodiment of the present disclosure.

### DETAILED DESCRIPTION OF THE PREFERRED EMBODIMENTS

The present disclosure will be illustrated in detail below with preferred embodiments and accompanying drawings. It should be noted that, the shape and scale of the diagrams disclosed in each embodiment below are intended to account for the technical features of the present disclosure, and are not intended to limit the aspects of the present disclosure on the practical implementation.

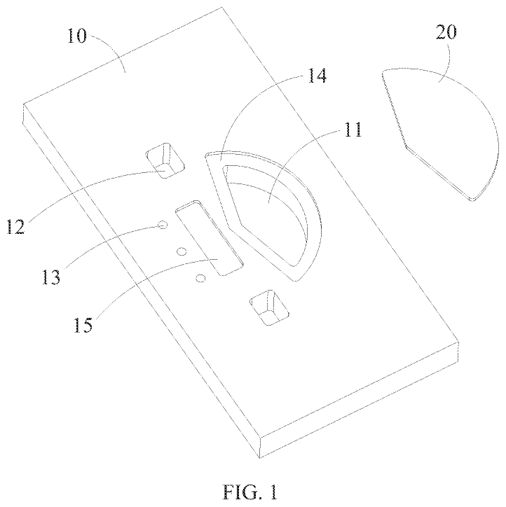

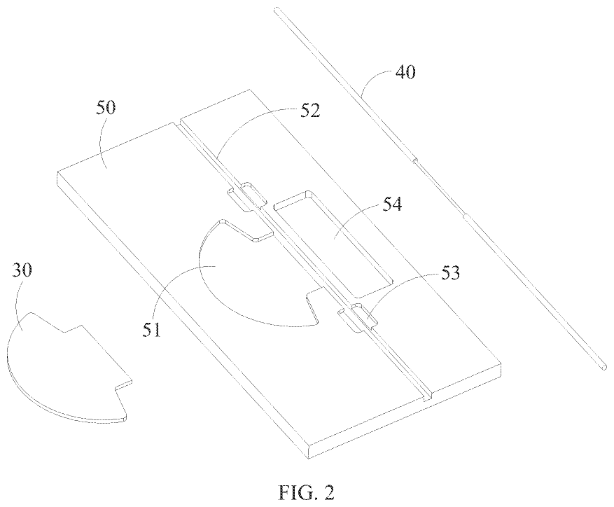

Referring to FIG. 1 and FIG. 2, FIG. 1 and FIG. 2 are stereoscopic diagrams of the biosensor chip in accordance with an embodiment of the present disclosure. As shown in FIG. 1 and FIG. 2, a biosensor chip of the present disclosure is substantially composed of a top cover 10, a plasma separation membrane 20, a conjugate pad 30, an optical fiber 40, and a bottom cover 50. Wherein the top cover 10 is provided with a sample loading slot 11 for injecting blood sample, a glue injection hole 12 for perfusion of glue, and a plurality of vent hole 13, and these structures are all configured as through holes penetrating through the top cover 10. Further, the top cover 10 further includes a separation membrane groove 14 for disposing the plasma separation membrane 20, and a flow channel 15 for connecting an optical fiber channel 52 and a waste liquid tank 54 after being combined with the bottom cover 50. As shown in the drawings, the plasma separation membrane 20 may be disposed to have a same shape as the sample loading slot 11 but larger than the sample loading slot 11, while the separation membrane groove 14 may be configured as a groove matching the size of the plasma separation membrane 20, so that the plasma separation membrane 20 can be embedded therein.

Corresponding to the top cover 10, the bottom cover is provided with a conjugate pad groove 51 for disposing the conjugate pad 30, an optical fiber channel 52 for disposing the optical fiber 40, a glue injection groove 53 corresponding to the position of glue injection hole 12 in the top cover 10 for disposing the glue, and a waste liquid tank 54. Wherein the position of the conjugate pad groove 51 corresponds to the sample loading slot 11 in the top cover 10 and is disposed on one side of the optical fiber channel 52, while being connected to the optical fiber channel 52, so that the sample liquid passing through the plasma separation membrane 20 and the conjugate pad 30 can flow into the optical fiber channel 52 and contact with the optical fiber 40. The glue injection groove 53 corresponds to the position of the glue injection hole 12, and partially overlaps with the optical fiber channel 52, for example, being disposed at both ends of the optical fiber channel 52 for perfusion of glue after the biosensor chip is assembled. The position of the waste liquid tank 54 is disposed at the other side of the optical fiber channel 52 opposite to the conjugate pad groove 51, so that the vent hole 13 can be located above the waste liquid tank 54 after the biosensor chip is assembled. As shown in the drawings, the conjugate pad 30 may be configured to have a shape similar to the plasma separation membrane 20 but larger than the plasma separation membrane 20, and the conjugate pad groove 51 may be configured to match the size of the conjugate pad 30, so that the conjugate pad 30 can be embedded therein.

The plasma separation membrane 20 may be a disposable paper-based plasma separation membrane, which is capable of separating plasma from blood sample in non-laboratory and other environments where centrifuge is not suitable. The material of plasma separation membrane 20 is polysulfone, polyethersulfone or glass fiber with porous structure. Compared with other materials, since the material of glass fiber is sharper, glass fiber may cause rupture of red blood cells and hemolysis. On the other hand, materials such as polysulfone and polyethersulfone are relatively soft, so the risk of hemolysis is lower than that of glass fiber. The porous structure of the membrane allows the blood cells in the whole blood to be captured in larger pores (˜100 μm) at the upper side without hemolysis, while the plasma flows down into smaller pores (˜2 μm) at the lower side of the membrane, and thus plasma will be simply and effectively separated from blood in two minutes without centrifugation.

The conjugate pad 30 may be made of nonabsorbable glass fiber, and the nanoparticles modified by the detection probe can be fixed on the conjugate pad 30 and kept stable. When the liquid sample is introduced into the conjugate pad 30, the liquid sample will dissolve the nanoparticles modified by the detection probe from the conjugate pad 30, and bind with the target analyte molecules in the sample which are bound by the nanoparticles modified by the detection probe. Wherein the analytes may include DNA, RNA, protein, small molecule or antibody, but the present disclosure is not limited thereto. In addition, the following will use gold nanoparticles as example for illustration, but the present disclosure is not limited thereto.

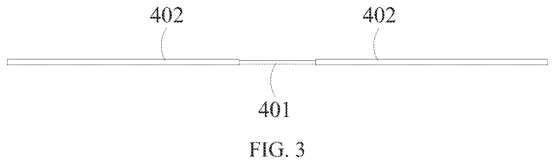

Subsequently, referring to FIG. 3, FIG. 3 is an enlarged view of the optical fiber 40 in FIG. 2. The optical fiber 40 used in the present disclosure may be made of glass and plastic, as shown in FIG. 3, a basic structure of the optical fiber 40 is composed of an optical fiber core 401 and cladding 402, and optical fiber core 401 is made of glass (silicon dioxide) or plastic material with a high refractive index, which may keep the light transmitted in the core layer; the cladding 402 on the outside is a cladding with a lower refractive index, which may be made of silicon dioxide or plastic, and the outermost coating is made of plastic material, which can protect the optical fiber form damage. In the present disclosure, the surface of the optical fiber core 401 is modified by a capture probe capable of capturing an analyte molecule. Wherein the detection probe and the capture probe are respectively binding with different sites of the analyte molecule. For example, if the analyte to be detected is DNA^{T}, DNA^{D} (detection probe) may be modified on the surface of gold nanoparticles, and DNAs (capture probe) may be modified on the surface of optical fiber core 401, so that when DNA^{T} exists in the sample, DNA^{D} on the surface of the gold nanoparticles and DNAs on the surface of the optical fiber core 401 can specifically bind with DNA^{T} at different sites, so that the gold nanoparticles are attached on the surface of the optical fiber core 401 to form a sandwich-like nanocomplex of capture probe-analyte-detection probe modified gold nanoparticle. Therefore, the optical fiber core 401 is preferably located in a section of the optical fiber corresponding to the position where the conjugate pad groove 51 and the optical fiber channel 52 are connected, so that the analyte molecules which are bound by the detection probe modified gold nanoparticles can follow the flow of the sample and contact the capture probe on the surface of the optical fiber core 401.

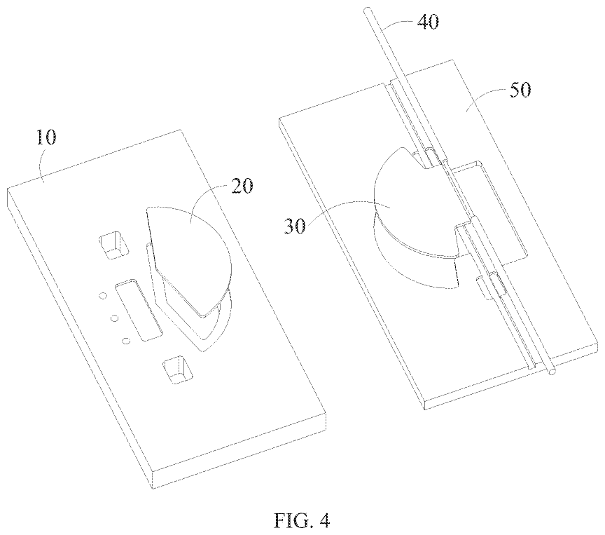

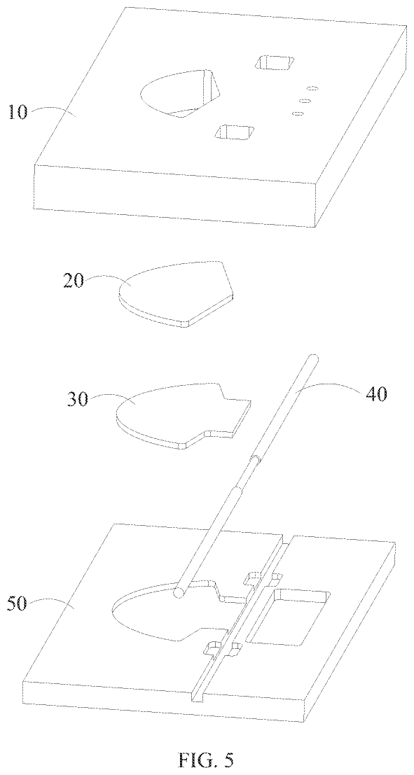

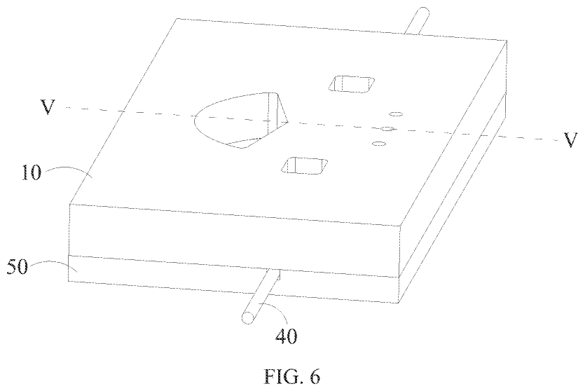

Referring to FIG. 4 to FIG. 6, FIG. 4 to FIG. 6 illustrate the actual assembly state of the biosensor chip in accordance with an embodiment of the present disclosure. As shown in the drawings, the plasma separation membrane 20 is assembled to the top cover 10, while the conjugate pad 30 and optical fiber 40 are assembled to the bottom cover 50. Therefore, when the top cover 10 and bottom cover 50 are combined, as shown in FIG. 5, the order from top to bottom will be top cover 10, plasma separation membrane 20, conjugate pad 30 and bottom cover 50, and finally assembled into the biosensor chip shown in FIG. 6. In addition, the optical fiber 40 may also be inserted into the optical fiber channel 52 after the top cover and the bottom cover 50 are combined, and finally inject glue (AB glue) into the glue injection hole 12 to complete the assembly of the biosensor chip.

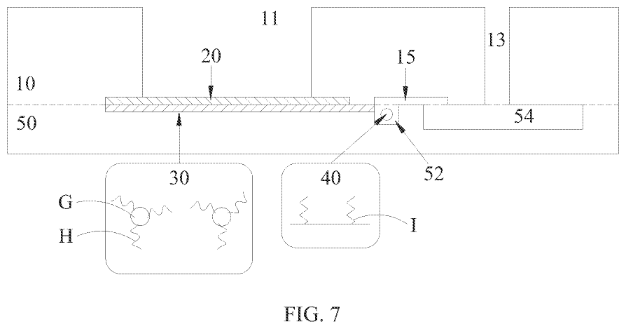

Further, referring to FIG. 7, FIG. 7 is a schematic cross-sectional view taken along the V-V line in FIG. 6. As shown in the drawing, when the blood sample is loaded into the sample loading slot 11, the large cells such as red blood cells and white blood cells are first blocked by the plasma separation membrane 20, so that the plasma enters the conjugate pad 30, and the gold nanoparticles G modified by the detection probe H (G@H) are dissolved, while an analyte molecules X in the plasma may bind with the detection probe H to form a nanocomplex G@H—X, and then flow into the optical fiber channel 52 to contact with the optical fiber 40, so that the analyte molecule X in the plasma may bind with the capture probe I on the optical fiber 40 (optical fiber core 401) to form a sandwich-like nanocomplex G@H—X—I, the optical fiber channel 52 and conjugate pad groove 51 here may be designed to have a height difference, so that the liquid is able to flow automatically. After that, the waste liquid may enter the waste liquid tank 54 through the flow channel 15.

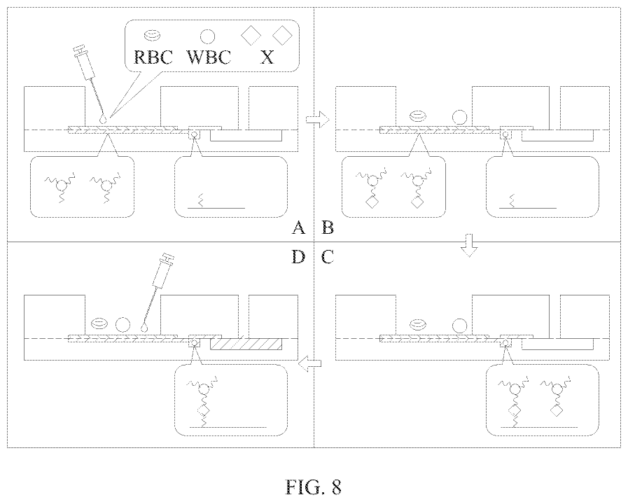

Referring to FIG. 8 for specific steps of operation, first, as shown in A of FIG. 8, the diluted blood sample is loaded into the sample loading slot 11, the blood sample includes, for example, red blood cells RBC, white blood cells WBC and target analyte X; then, as shown in B of FIG. 8, the red blood cells RBC and white blood cells WBC are blocked by the plasma separation membrane 20, only plasma enters the conjugate pad 30, and the gold nanoparticles G modified by the detection probe H (G@H) are dissolved out, while one analyte molecule X binds with one detection probe H to form a nanocomplex G@H—X; then, as shown in C of FIG. 8, the analyte molecule X in the plasma binds with the detection probe modified gold nanoparticles G to form the nanocomplex G@H—X and flows into the optical fiber channel 52 together and reacts for a specific time (such as 15 minutes), so that the analyte molecule X can bind with the capture probe I on the optical fiber 40 (optical fiber core 401), and forms a sandwich-like nanocomplex G@H—X—I; finally, as shown in D of FIG. 8, a buffer solution PBS is injected into the sample loading slot 11 to wash the unreacted analyte molecules X or G@H—X nanocomplexes into the waste liquid tank 54, leaving the sandwich-like nanocomplex G@H—X—I binding on the optical fiber 40 only, and then according to the sensing method of the FOPPR biosensor chip, 530 nm green light is incident into the optical fiber 40, and the concentration of the analyte X is analyzed from the light signal.

In summary, a biosensor chip of the present disclosure is combined with a plasma separation membrane and a conjugate pad, so one only need to inject diluted blood sample to separate plasma from the blood sample, and the analyte in the plasma may be detected. Therefore, the present disclosure provides a simpler and faster way to analyze blood samples.

The present disclosure has been described above with preferred embodiments, but the description of preferred embodiments is not intended to limit the scope of the present disclosure. Many modifications and variations will be apparent to those having ordinary skill in the art without departing from the spirit and scope of the present disclosure. Therefore, the scope of the present disclosure should be defined by the appended claims.

What is claimed is:

1. A biosensor chip, comprising:
a top cover provided with a sample loading slot for injecting a blood sample, a glue injection hole, and a vent hole;
a plasma separation membrane overlapping the sample loading slot of the top cover;
a conjugate pad overlapping the plasma separation membrane;
an optical fiber; and
a bottom cover provided with a conjugate pad groove for disposing the conjugate pad, an optical fiber channel for disposing the optical fiber, a glue injection groove corresponding to a position of the glue injection hole in the top cover for disposing a glue, and a waste liquid tank,
wherein, the conjugate pad groove is connected to the optical fiber channel, the glue injection groove is partially overlapped with the optical fiber channel, and the vent hole is located above the waste liquid tank,
the top cover further includes a separation membrane groove for disposing the plasma separation membrane, and a flow channel for connecting the optical fiber channel and the waste liquid tank.

2. The biosensor chip of claim 1, wherein the conjugate pad includes nanoparticles modified by a detection probe, which is capable of binding with an analyte in the blood sample.

3. The biosensor chip of claim 2, wherein the detection probe includes one selected from a group consisting of antibodies, nucleic acid aptamers, peptides, hormone receptors, lectins, carbohydrates, chemical recognition molecules, deoxyribonucleic acid, ribonucleic acid, and nucleic acid aptamers.

4. The biosensor chip of claim 2, wherein the nanoparticles are gold nanoparticles.

5. The biosensor chip of claim 2, wherein the analyte include DNA, RNA, protein, small molecule, or antibody.

6. The bio sensor chip of claim 2, wherein the optical fiber includes an optical fiber core which is an exposed section of the optical fiber, a surface of the optical fiber core is modified by a capture probe capable of capturing the analyte molecules bound by the nanoparticles modified by the detection probe, and the analyte molecules bound to the nanoparticles modified by the detection probe forms a sandwich-like nanocomplex with the capture probe.

7. The bio sensor chip of claim 6, wherein the capture probe includes one selected from a group consisting of antibodies, nucleic acid aptamers, peptides, hormone receptors, lectins, carbohydrates, chemical recognition molecules, deoxyribonucleic acid, ribonucleic acid, and nucleic acid aptamers.

8. The biosensor chip of claim 6, wherein the optical fiber core is located in a section of the optical fiber corresponding to a position where the conjugate pad groove and the optical fiber channel are connected.

9. The biosensor chip of claim 1, wherein the plasma separation membrane includes polysulfone, polyethersulfone, or glass fiber.

10. The biosensor chip of claim 1, wherein the conjugate pad is made of nonabsorbable glass fiber.
